(19) United States
(12) Patent Application Publication
(10) Pub. No.: US 2018/0147331 A1
(43) Pub. Date: May 31, 2018

(54) ELECTRIC BREAST PUMP

(71) Applicant: MAMAWAY LICENSING CO., LTD., Apia (WS)

(72) Inventor: Kuei-Chen LIU, Taipei City (TW)

(21) Appl. No.: 15/622,932

(22) Filed: Jun. 14, 2017

(30) Foreign Application Priority Data
Nov. 25, 2016 (TW) 105138948

(51) Int. Cl. A61M 1/06; A61M 39/10

(52) U.S. Cl. CPC A61M 1/062; A61M 39/10; A61M 2205/3673; A61M 2205/368; A61M 2039/1022; A61M 2205/3337; A61M 2205/8206; A61M 2205/33

(57) ABSTRACT

The present invention provides an electric breast pump. The electric breast pump includes a conduit module, a suction module and a host. The conduit module includes a connector, a first conduit and a second conduit. The connector has an electric contact and an opening hole, and the connector is further disposed with a protrusion at the side of the connector on which the opening hole is formed. The first end of the first conduit connects to the electric contact. The first end of the second conduit connects to the opening hole. The suction module has an adapter body coupled with the connector to connect the suction module and the conduit module. The host connects to the second end of the first conduit and the second end of the second conduit. The host controls the suction module via the conduit module to extract and collect milk when the suction module contacts mother's breast.

### CROSS-REFERENCE TO RELATED APPLICATION

The present application claims benefit of priority to Taiwan Patent Application No. 105138948, filed on Nov. 25, 2016, the entire disclosure of which is hereby incorporated by reference herein.

### BACKGROUND OF THE INVENTION

### 1. Field of the Invention

The present invention relates to electric breast pumps, and, more specifically, to an electric breast pump having a conduit module connecting to a host and a suction module, and utilizing electrothermally heating and negative pressure to facilitate lactation.

### 2. Description of Related Art

Postpartum women usually need to breastfeed, but sometimes, they are too busy with household chores or work to personally breastfeed. Alternatively, when the postpartum women make too much milk, it is necessary to purchase a breast pump to help them collecting breast milk.

There are many kinds of breast pumps available in the market, and most of them can be generally classified into manual breast pumps or electric breast pumps. Generally speaking, electric breast pumps comprise a funnel for extracting milk, a bottle and a host connected with wires. The frequency or suction intensity of electric breast pumps is adjustable to simulate and match the sucking speed and strength of babies. However, it is a major problem how to disassemble, clean and then reassemble the relevant parts after each use of the electric breast pump. Many manufactures and developers in the related industry try to improve current products in the market because of the complicated steps of disassembly and reassembly.

Thus, there is an urgent need to develop an electric breast pump which is easy to disassemble and reassemble and is multi-functional.

### SUMMARY OF THE INVENTION

In order to solve the foregoing problems of the prior art, the present invention provides an electric breast pump, which comprises a conduit module, a suction module and a host. The conduit module comprises: a connector having an electric contact and an opening hole, and a protrusion is disposed at the side of the connector on which the opening hole is formed; a first conduit having a first end connected to the electric contact and a second end opposite the first end; and a second conduit having a first end connected to the opening hole and a second end opposite the first end. The suction module has an adapter body coupled with the connector for connecting the suction module and the conduit module; and the host connected with the second end of the first conduit and the second end of the second conduit. The host controls the suction module via the conduit module and thereby the suction module extracts and collects milk when the suction module contacts mother's breast.

In an embodiment, the present invention further comprises an electrothermal unit disposed in or on the adapter body of the suction module.

In an embodiment, the adapter body comprises a funnel, an engaging portion, and a connecting portion. The funnel has a first through hole, and wherein the electrothermal unit is embedded in the funnel; the engaging portion is provided in a direction opposite to a mouth of the funnel, and the engaging portion has a socket and a second through hole, wherein the socket is electrically connected to the electrothermal unit and the electric contact is electrically connected to the socket, and the second through hole corresponds to the opening hole; and the connecting portion is provided below the funnel and the engaging portion, and the connecting portion has a third through hole, wherein the third through hole is interconnected with the first through hole and the second through hole; the suction module further comprises a diaphragm piece and a valve, wherein the diaphragm piece is disposed in the second through hole to separate the opening hole from the second through hole; and the valve is disposed in the third through hole. The connector connects to the adapter body by engaging the protrusion with the engaging portion.

In an embodiment, the suction module further comprises a containing bottle having a lip detachably coupled with the connecting portion of the adapter body.

In an embodiment, the host comprises: an inner chamber disposed within the host, the inner chamber having an accommodating space; a negative pressure generating unit disposed within the host for generating the negative pressure; a controlling unit disposed on the surface of the host for controlling the operation of the negative pressure generating unit and the electrothermal unit; and an electric power module joined to the host for supplying electric power to the negative pressure generating unit, the electrothermal unit and the controlling unit.

In an embodiment, the host further comprises a receiving port, and wherein the second end of the first conduit connects to the controlling unit via the receiving port and the second end of the second conduit connects to the negative pressure generating unit via the receiving port.

In an embodiment, the present invention further comprises a lower cover, and wherein the inner chamber has an opening connected to the accommodating space, and the lower cover openably and closably covers the opening of the inner chamber.

In an embodiment, the present invention further comprises a switch disposed on the lower cover for opening or closing the lower cover.

In an embodiment, the electric power module has an electric power transmission unit for receiving electric power from an external power source for supplying electric power to the electric power module.

In an embodiment, the electric power module further comprises a battery unit which is managed by the electric power module to supply electric power or be recharged by the electric power transmission unit and then supply electric power.

In an embodiment, the host further comprises a display unit disposed on the surface of the host for displaying the on/off state of the electric power module, the operation state of the negative pressure generating unit or the heating state of the electrothermal unit.

In an embodiment, the controlling unit is a knob and/or a button, and wherein the knob is used to switch on the electric power module or control the operation of the negative pressure generating unit, and the button is used to control the operation of electrothermal unit.

In an embodiment, the second conduit is arranged side by side with the first conduit.

Compared to the prior art, the electric breast pump of the present invention comprises a host, a suction module and a conduit module, wherein the host has an inner chamber to accommodate the conduit module and there is an electrothermal unit embedded in the funnel which would contact mother's body. Besides, the conduit module is connected to the host and the suction module. Consequently, the electrothermal unit enables the electric breast pump of the present invention to be heated up, thereby keeping mother's breast warm while using the electric breast pump of the present invention.

### BRIEF DESCRIPTION OF THE DRAWINGS

FIG. 1 is a schematic view of the conduit module of the electric breast pump according to an exemplary embodiment of the present invention.

FIG. 2 is a schematic view of the connector of the conduit module of the electric breast pump according to an exemplary embodiment of the present invention.

FIG. 3 is a schematic view showing that the suction module is coupled with the conduit module of the electric breast pump according to an exemplary embodiment of the present invention.

FIG. 4 is a cross-section view showing that the suction module coupled with the conduit module of the electric breast pump according to an exemplary embodiment of the present invention.

FIG. 5 is a schematic view of the adapter body of the suction module of the electric breast pump according to an exemplary embodiment of the present invention.

FIG. 6 is a first schematic of the host of the electric breast pump according to an exemplary embodiment of the present invention.

FIG. 7 is a second schematic of the host of the electric breast pump according to an exemplary embodiment of the present invention.

FIG. 8 is a third schematic of the host of the electric breast pump according to an exemplary embodiment of the present invention.

FIG. 9 is a functional block diagram of the assembly of the electric breast pump according to an exemplary embodiment of the present invention.

### DETAILED DESCRIPTION OF THE EMBODIMENTS

The present invention is described by the following specific embodiments. Those with ordinary skills in the arts can readily understand other advantages and functions of the present invention after reading the disclosure of this specification. The present disclosure may also be practiced or applied with other different implementations. Based on different contexts and applications, the various details in this specification can be modified and changed without departing from the spirit of the present disclosure.

The embodiments are described below in order to explain the present general inventive concept while referring to the figures. Also, while describing the present general inventive concept, detailed descriptions about related well-known functions or configurations that may diminish the clarity of the points of the present general inventive concept are omitted. It should be noted that the structures, ratios, sizes shown in the drawings appended to this specification are to be construed in conjunction with the disclosure of this specification in order to facilitate understanding of those skilled in the art. They are not meant, in any ways, to limit the implementations of the present invention, and therefore have no substantial technical meaning. Without affecting the effects created and objectives achieved by the present invention, any modifications, changes or adjustments to the structures, ratio relationships or sizes, are to be construed as fall within the range covered by the technical contents disclosed herein. Meanwhile, terms, such as “upper”, “inner”, “external”, “bottom”, “one”, “a” and the like, are for illustrative purposes only, and are not meant to limit the range implementable by the present invention. Also, when a part “includes” or “comprises” an element, unless there is a particular description contrary thereto, the part can further include other elements, not excluding the other elements. In the following description, terms such as “unit” and “module” indicate a unit to process at least one function or operation, wherein the unit and the module may be embodied as hardware or software or embodied by combining hardware and software. Any changes or adjustments made to their relative relationships, without modifying the substantial technical contents, are also to be construed as within the range implementable by the present invention.

Hereinafter, one or more exemplary embodiments of the present general inventive concept will be described in detail with reference to accompanying drawings.

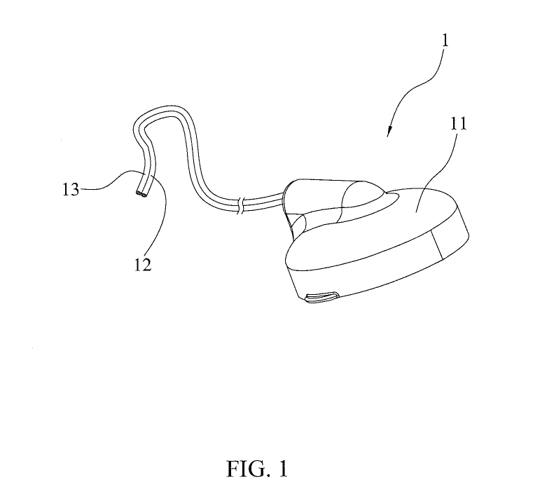

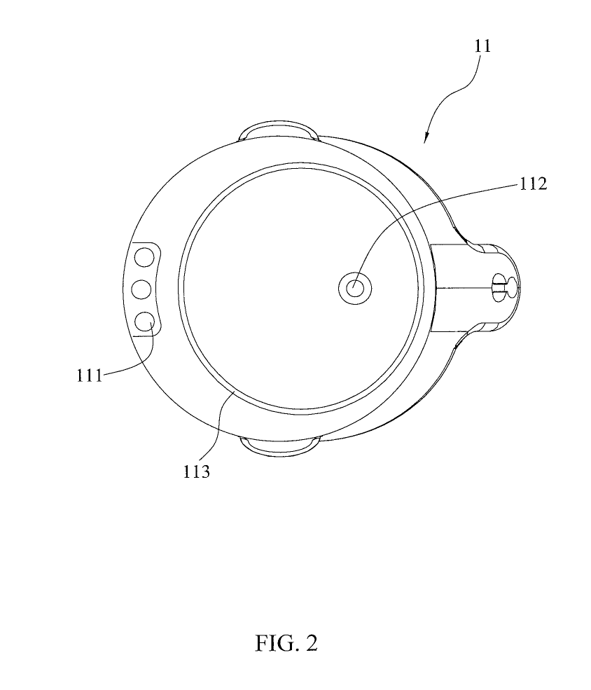

The present invention provides an electric breast pump, which comprises a conduit module 1, a suction module 2 and a host 3. Refer to FIGS. 1 and 2. FIG. 1 is a schematic view of the conduit module of an electric breast pump according to an exemplary embodiment of the present invention. FIG. 2 is a schematic view of the connector of a conduit module of an electric breast pump according to an exemplary embodiment of the present invention. According to the exemplary embodiment of the present invention, the conduit module 1 comprises a connector 11, a first conduit 12 and a second conduit 13. The connector 11 has an electric contact 111 and an opening hole 112. The connector 11 further has a protrusion 113 disposed at the side of the connector where the opening hole 112 is formed. The first conduit 12 has a first end and a second end opposite the first end and the first end of the first conduit 12 may be connected to the electric contact 111. The second conduit 13 has a first end and a second end opposite the first end and the first end of the second conduit 13 may be connected to the opening hole 112. The second conduit 13 may be arranged side by side with the first conduit 12.

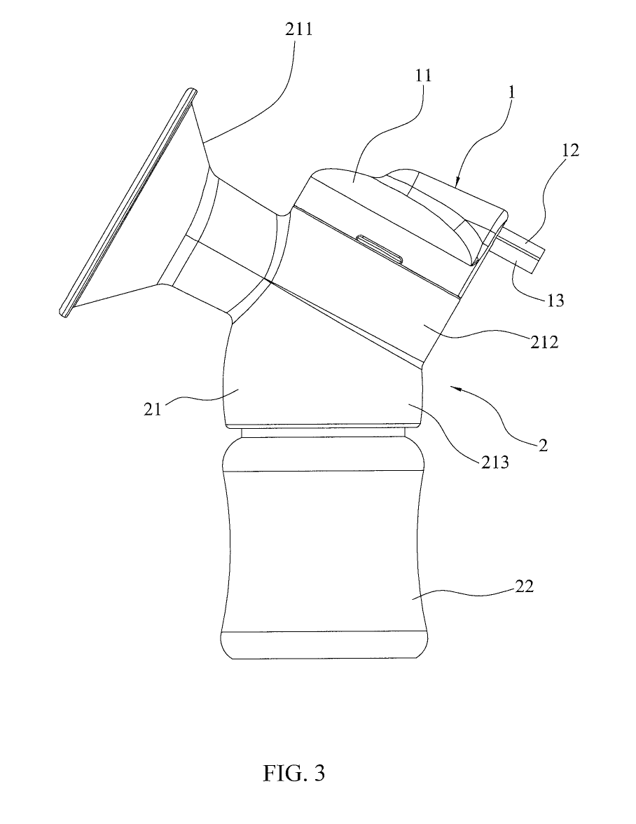

Refer to FIG. 3 which is a schematic view showing that a suction module coupled with the conduit module of an electric breast pump according to an exemplary embodiment of the present invention. As shown in the drawings, the suction module 2 has an adapter body 21. The adapter body 21 may comprise a funnel 211, an engaging portion 212 and a connecting portion 213. The engaging portion 212 may be provided in a direction opposite to a mouth of the funnel 211. The connecting portion 213 may be provided below the funnel 211 and the engaging portion 212. The connector 11 of the conduit module 1 may connect to the adapter body 21 by engaging the protrusion 113 with the engaging portion 212 (with reference to FIG. 2).

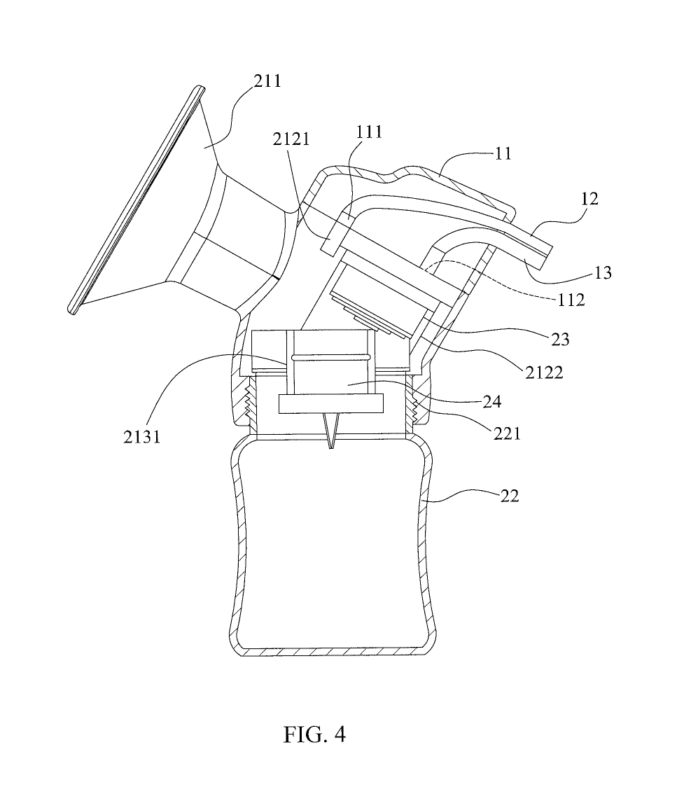

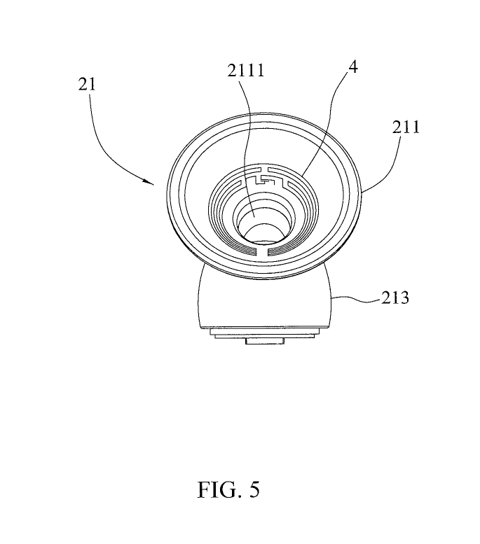

More details of the internal structure of the suction module 2 and the conduit module 1 of an exemplary embodiment of the present invention are illustrated in FIGS. 4 and 5. FIG. 4 is a cross-section view showing that a suction module coupled with a conduit module of the electric breast pump according to an exemplary embodiment of the present invention. FIG. 5 is a schematic view of an adapter body of a suction module of the electric breast pump according to an exemplary embodiment of the present invention. The funnel 211 may have a first through hole 2111 (with reference to FIG. 5) and the engaging portion 212 may have a socket 2121 and a second through hole 2122. When the connector 11 is connected to the engaging portion 212, the electric contact 111 may be connected to the socket 2121 and the second through hole 2122 may correspond to the opening hole 112. The connecting portion 213 may have a third through hole 2131. Besides, the suction module 2 may further comprise a diaphragm piece 23 and a valve 24. The diaphragm piece 23 may be disposed in the second through hole 2122 so as to separates the opening hole 112 from the second through hole 2122. The valve 24 may be disposed in the third through hole 2131.

In this embodiment, the suction module 2 may further comprise a containing bottle 22 having a lip 221. The lip 221 of the containing bottle 22 may be detachably coupled with the connecting portion 213 of the adapter body 21. The containing bottle 22 can be used to contain mother's milk extracted by the suction module 2.

Further refer to FIG. 5. According to the present embodiment, the suction module 2 may further comprise an electrothermal unit 4. The electrothermal unit 4 may be disposed in or on the adapter body 21. More specifically, the electrothermal unit 4 may be but not limited to, for example, flexible heater, such as a film heater made of polyimide or silicone. The electrothermal unit may be embedded in the funnel 211 of the adapter body 21 by means of, for example, the plastic injection moulding technique. In addition, according to some exemplary embodiments of the present invention, known far-infrared material can be added into or be coated on the inside wall of the funnel 211. The far-infrared wavelength emitted by the far-infrared material is similar to that emitted by the human body, so the radiant heat emitted by the far-infrared material is able to penetrate deeper beneath the skin. Therefore, the heating energy generated by the electrothermal unit 4 can be efficiently conducted to the human body to warm the human body. The temperature of the electrothermal unit 4 may be maintained at about 38 degrees Celsius (which may be similar to or slightly higher than the nom al human body temperature). The electrothermal unit 4 may be electrically connected with the socket 2121 of the engaging portion 212. When the electric contact 111 of the connector 11 is coupled with (electrically connected to) the socket 2121 of the engaging portion 212, electric powers may be transmitted to the electrothermal unit 4 through the first conduit 12 to heat up the mouth of the funnel 211. The temperature of the funnel 211 is close to the human body temperature, and thereby enhancing the comfort of the user using the electric breast pump of the present invention.

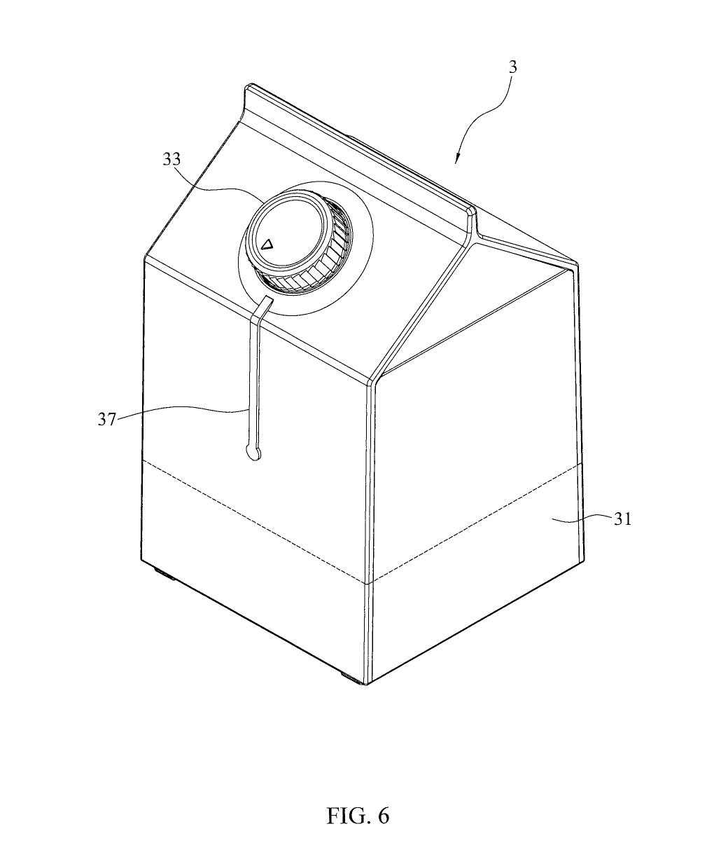

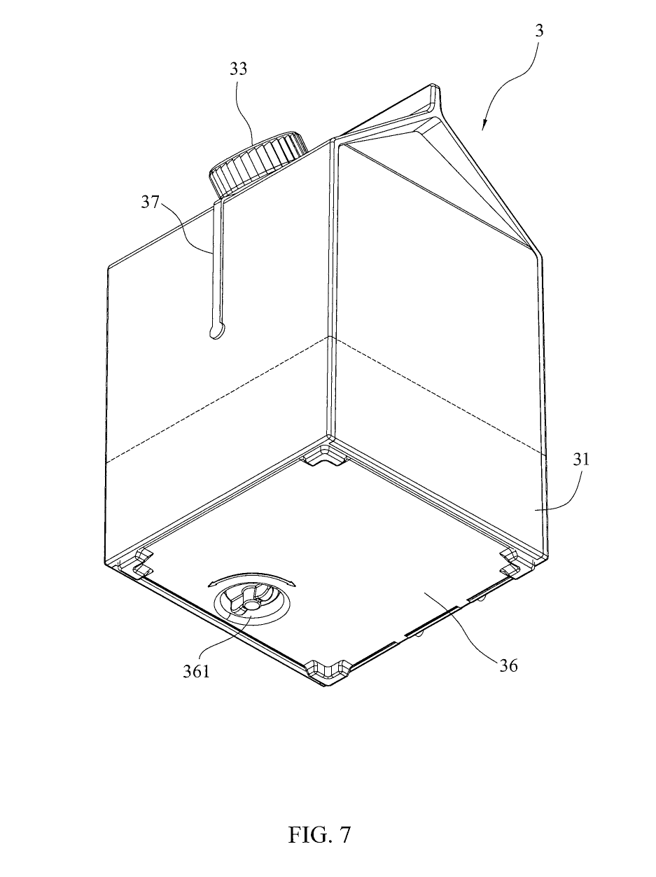

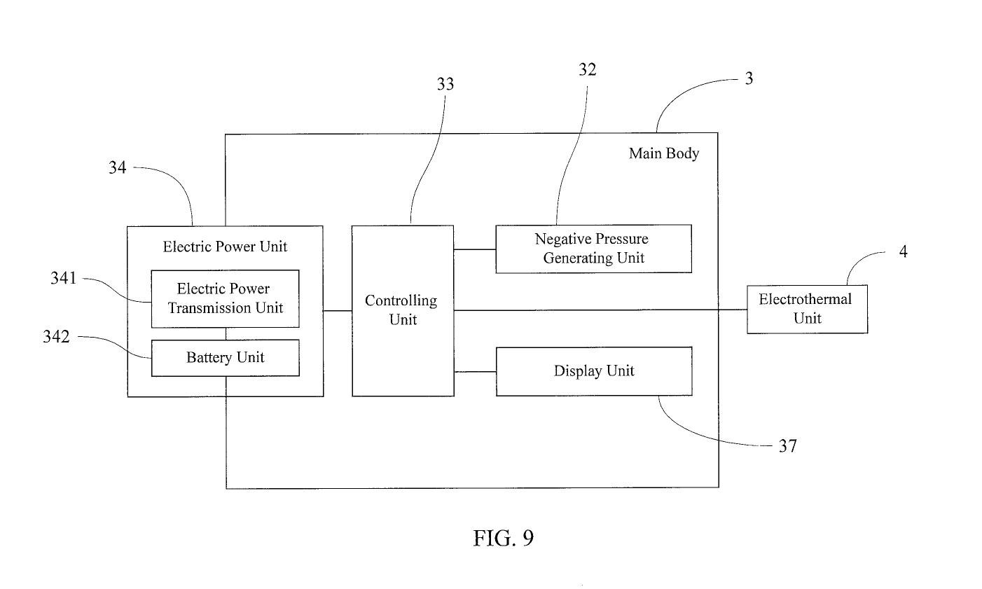

Refer to FIGS. 6 to 9. FIGS. 6 to 8 are schematics of three views of the host of an electric breast pump according to an exemplary embodiment of the present invention. FIG. 9 is a functional block diagram of the assembly of an electric breast pump according to an exemplary embodiment of the present invention. As shown in the drawings, the host 3 may comprise an inner chamber 31 disposed within the host 3, a negative pressure generating unit 32 disposed within the host 3, a controlling unit 33 disposed on the surface of the host 3 for controlling the operation of the negative pressure generating unit 32 and the electrothermal unit 4, and an electric power module 34 joined to the host 3 for supplying electric power to the negative pressure generating unit 32, the electrothermal unit 4 and the controlling unit 33. Preferably, the inner chamber 31 may be partitioned into a plurality of accommodating spaces to accommodate components of the conduit module 1 and suction module 2, such as the diaphragm piece 23, the connector 11, the first conduit 12 and the second conduit 13. The bottom of the host 3 may have a lower cover 36. The inner chamber 31 has an opening (not shown in the figures) connected to the accommodating space. As shown in FIG. 2, the lower cover 36 is used to cover the opening of the inner chamber 31. Furthermore, the lower cover 36 may further have a switch 361 disposed thereon. The lower cover 36 may openably/closably cover the opening of the inner chamber 31 by the switch 361. The negative pressure generating unit 32 may be, for example, a pump for generating negative pressures or vacuum in the suction module 2 via the second conduit 13 thereby to generate a suction force to extract milk when the funnel 211 is attached to mother's breast.

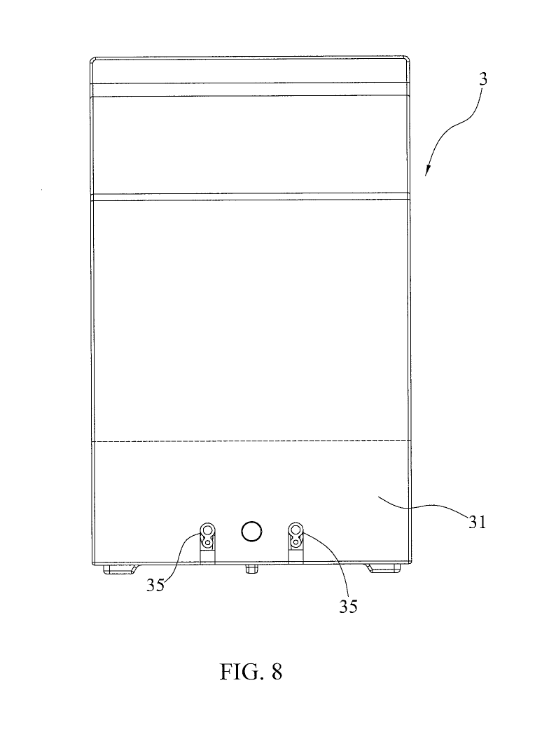

As shown in FIGS. 8 and 9, the electric power module 34 may have an electric power transmission unit 341. The electric power transmission unit 341 may be a built-in or external transformer for connecting the electric power module 34 and an external power source. The electric power module 34 may be used to supply electric power by the electric power transmission unit 341 receiving an external electric power from mains electricity or a mobile power pack. In other exemplary embodiments, the electric power module 34 may further comprise a battery unit 342. The battery unit 342 may be controlled or managed by the electric power module 34 to supply electric power. The battery unit 342 may be a non-rechargeable battery, such as a dry battery for supplying power to the negative pressure generating unit 32, the electrothermal unit 4, and the control unit 33. In another embodiment, the battery unit 342 may be a rechargeable battery built in the host 3. In these embodiments, the battery unit 342 may be recharged by the power transmission unit 341 and then repeatedly supply power to the negative pressure generating unit 32, the electrothermal unit 4, and the control unit 33.

The conduit module 1 is used to connect the host 3 and the suction module 2. When the negative pressure generating unit 32 is running, the negative pressure can be generated in the suction module 2 through the conduit module 1, and thereby generating suction force to extract and then collect milk when contacting mother's breast. In an exemplary embodiment of the present invention, a negative pressure generating unit 32 and a conduit module 1 are applied to one breast. In the case of the present invention, there may be usually a pair of negative pressure generating units 32 and a pair of conduit modules 1. Besides, in another exemplary embodiment, the host 3 may further comprise at least one receiving port 35 (as shown in FIG. 8) which the first conduit 12 and the second conduit 13 may be fixed on. The second end of the first conduit 12 may be connected to the control unit 33 via the receiving port 35, and the second end of the second conduit 13 may be connected to the negative pressure generating unit 32 via the receiving port 35. The required electric power for the electrothermal unit 4, the control unit 33, and the negative pressure generating unit 32 may all be supplied by the electric power module 34.

In an exemplary embodiment of the present invention, the electric breast pump may further comprise a display unit 37 disposed on the surface of the host 3. The display unit 37 may be, for example, a light-emitting column having a scale. The display unit 37 displays the on/off state of the electric power module 34 and the heating state of the electrothermal unit 4. The controlling unit 33 is a knob and/or a button. In this embodiment shown in FIG. 6, when the user turns on the electric power module 34, the bottom light signal of the display unit 37 may flash to indicate that the power is on. Then the electric contact 111 of the connector 11 of the conduit module 1 is electrically connected to the socket 2121 of the engaging portion 212 of the suction module 2 and in this stage, the bottom light signal of the display unit 37 keeps on. The light-emitting column would rise up to a predetermined length which means the temperature of the electrothermal unit 4 is rising to warm the suction module 2 up, which is considered to be a “preheating procedure”. In some embodiment of the present invention, the control unit 33 may include a button. The user can directly press the button to cancel the preheating procedure. If the preheating procedure is completed, the light-emitting column will rise to another predetermined length which means the preheating procedure has been completed and the suction module 2 is available to be used. In addition, the user can rotate the knob of the controlling unit 33 to adjust the strength of the negative pressure generating unit 32 to vary the suction force for extracting milk.

In some embodiments of the present invention, the negative pressure generating unit 32 may have two modes of operation to extract milk. The first mode may be a “lactation promotion” mode with high-frequency and low-intensity. The first mode can speed up the lactation. The second mode is a “stable collection” mode with low-frequency and high-intensity. It should be noted that the frequencies of the two modes are unchangeable, which means the electric breast pump of the exemplary embodiment of the present invention has only two fixed frequencies which are high and low. However, the user can adjust the strength of the negative pressure generating unit 32 by rotating the controlling unit 33. The aforesaid operating modes are merely illustrative, and in some other different embodiments of the present invention, the negative pressure generating unit 32 may be set to have other operating modes.

Refer to FIGS. 3 and 4 again. The conduit module 1 and the suction module 2 are connected to each other through the connector 11 and the engaging portion 212. The electric contact 111 is electrically connected to the socket 2121. The protrusion 113 engages with the second through hole 2122. The electric power module 34 supplies electric power to the electrothermal unit 4 through the first conduit 12 to heat the electrothermal unit 4. The negative pressure generating unit 32 transmits air to the second through hole 2122 through the second conduit 13 via the opening hole 112. Although the first through hole 2111, the second through hole 2122 and the third through hole 2131 of the adapter body 21 are interconnected with each other, the diaphragm piece 23 has been provided in the second through hole 2122 to substantially block the air flow into the chamber formed by the first through holes 2111, the second through holes 2122 and the third through holes 2131. When the funnel 211 contacts mother's breast, the controlling unit 33 controls the negative pressure generating unit 32 to generate a pressure difference to change the shape of the diaphragm piece 23 and thereby changing the volume and the pressure of the chamber to generate a suction force to extract milk. Furthermore, the extracted milk flows into the chamber via the first through hole 2111, and does not enter the second conduit 13 through the second through hole 2122 due to the partition of the diaphragm piece 23. The milk enters the containing bottle 22 via the third through hole 2131 through the valve 24, and is stored therein. The milk passes through the valve 24 by gravity. The valve 24 can prevent the milk in the containing bottle 22 from being sucked out.

In summary, the electric breast pump of the present invention comprises a conduit module which is capable of connecting the host and transmiting electric power and air and an electrothermal unit embedded in the funnel of the suction module for heating. With regard to mother, it is quite a convenient and practical design

The above embodiments are only used to describe the principles of the present invention, but not to limit the scope of the present invention in any way. The above embodiments can be modified or changed by anyone with ordinary skill in the art without departing from the scope of the disclosure. Accordingly, all modifications and variations completed by those with ordinary skill in the art should fall within the scope of present invention defined by the appended claims. The scope of the present invention as defined in the following appended claims.

1. An electric breast pump, comprising:
a conduit module, comprising:
a connector having an electric contact and an opening hole, and a protrusion is further disposed at the side of the connector where the opening hole is formed;
a first conduit having a first end and a second end opposite the first end, wherein the first end of the first conduit is connected to the electric contact; and
a second conduit having a first end and a second end opposite the first end, wherein the first end of the second conduit is connected to the opening hole;
a suction module having an adapter body coupled with the connector for connecting the suction module and the conduit module; and
a host connected with the second end of the first conduit and the second end of the second conduit,
wherein the suction module is controlled by the host via the conduit module and thereby the suction module extracts and collects milk when contacting mother's breast.

2. The electric breast pump of claim 1, further comprising an electrothermal unit disposed in or on the adapter body of the suction module.

3. The electric breast pump of claim 2, wherein:
the adapter body comprises:
a funnel having a first through hole, and the electrothermal unit is embedded in the funnel;
an engaging portion provided in a direction opposite to a mouth of the funnel, wherein the engaging portion has a socket and a second through hole, wherein the socket is electrically connected to the electrothermal unit; and the electric contact is electrically connected to the socket, and the second through hole corresponds to the opening hole; and
a connecting portion provided below the funnel and the engaging portion, and the connecting portion has a third through hole, wherein the third through hole is interconnected with the first through hole and the second through hole; and
the suction module further comprises:
a diaphragm piece disposed in the second through hole for separating the opening hole from the second through hole; and
a valve disposed in the third through hole,
wherein the connector connects to the adapter body by engaging the protrusion with the engaging portion.

4. The electric breast pump of claim 3, wherein the suction module further comprises a containing bottle having a lip, wherein the lip is detachably coupled with the connecting portion of the adapter body.

5. The electric breast pump of claim 2, wherein the host comprises:
an inner chamber disposed within the host, and wherein the inner chamber has an accommodating space;
a negative pressure generating unit disposed within the host for generating the negative pressure;
a controlling unit disposed on the surface of the host for controlling the operation of the negative pressure generating unit and the electrothermal unit; and
an electric power module joined to the host for supplying electric power to the negative pressure generating unit, the electrothermal unit, and the controlling unit.

6. The electric breast pump of claim 5, wherein the host further comprises a receiving port, and wherein the second end of the first conduit connects to the controlling unit via the receiving port and the second end of the second conduit connects to the negative pressure generating unit via the receiving port.

7. The electric breast pump of claim 5, further comprising a lower cover, wherein the inner chamber has an opening connected to the accommodating space, and the lower cover openably and closably covers the opening of the inner chamber.

8. The electric breast pump of claim 7, further comprising a switch disposed on the lower cover for opening or closing the lower cover.

9. The electric breast pump of claim 5, wherein the electric power module has an electric power transmission unit for receiving electric power from an external power source for supplying electric power to the electric power module.

10. The electric breast pump of claim 9, wherein the electric power module further comprises a battery unit, wherein the battery unit is managed by the electric power module to supply electric power, or to be recharged by the electric power transmission unit and then supply electric power.

11. The electric breast pump of claim 5, wherein the host further comprises a display unit disposed on the surface of the host for displaying the on/off state of the electric power module, the operation state of the negative pressure generating unit or the heating state of the electrothermal unit.

12. The electric breast pump of claim 5, wherein the controlling unit comprises a knob and a button, and wherein the knob is used to switch on the electric power module or control the operation of the negative pressure generating unit, and the button is used to control the operation of electrothermal unit.

13. The electric breast pump of claim 1, wherein the second conduit is arranged side by side with the first conduit.
